(19) United States
(12) Patent Application Publication
(10) Pub. No.: US 2025/0164902 A1
(43) Pub. Date: May 22, 2025

(54) ELECTROPHOTOGRAPHIC PHOTOSENSITIVE MEMBER, PROCESS CARTRIDGE, AND ELECTROPHOTOGRAPHIC APPARATUS

(71) Applicant: CANON KABUSHIKI KAISHA, Tokyo (JP)

(72) Inventors: MASASHI NISHI, Kanagawa (JP); TSUTOMU NISHIDA, Shizuoka (JP); AKIHIRO MARUYAMA, Shizuoka (JP); NORIFUMI MURANAKA, Kanagawa (JP)

(21) Appl. No.: 18/942,027

(22) Filed: Nov. 8, 2024

(30) Foreign Application Priority Data
Nov. 16, 2023 (JP) 2023-195280
Oct. 2, 2024 (JP) 2024-173827

(51) Int. Cl. G03G 5/05; C07C 211/54; C08G 63/672; G03G 5/04; G03G 5/06

(52) U.S. Cl. CPC G03G 5/056; C07C 211/54; C08G 63/672; G03G 5/04; G03G 5/0659

(57) ABSTRACT

Provided is an electrophotographic photosensitive member including: a support; and a photosensitive layer. The photosensitive layer contains a binder resin, a charge generating material, an electron transporting material, and a hole transporting material. The binder resin contains a polyarylate resin having a particular structural unit. The hole transporting material contains a particular compound.

### BACKGROUND OF THE INVENTION

### Field of the Invention

The present invention relates to an electrophotographic photosensitive member, a process cartridge including the electrophotographic photosensitive member, and an electrophotographic apparatus including the electrophotographic photosensitive member.

### Description of the Related Art

In recent years, there has been a demand for an electrophotographic apparatus having a longer service life and being capable of forming an image of higher image quality, and it has been desired to provide an apparatus having high stability of image quality of an image to be output even at the time of repeated use (hereinafter sometimes referred to as “after endurance”).

An organic electrophotographic photosensitive member (hereinafter sometimes simply referred to as “electrophotographic photosensitive member” or “photosensitive member”) containing an organic photoconductive material (charge generating material) is used as an electrophotographic photosensitive member to be mounted on an electrophotographic apparatus or a process cartridge to be mounted on an electrophotographic apparatus. In the recent electrophotographic apparatus, there has been proposed a technology for improving the durability of various components in order to respond to the above-mentioned longer service life.

For example, in each of Japanese Patent Application Laid-Open Nos. 2022-49733 and 2023-19706, there has been proposed a photosensitive member that uses a polyarylate resin (PAR) excellent in wear resistance as a binder resin in a photosensitive layer.

According to investigations made by the inventors of the present invention, in the photosensitive member described in each of Japanese Patent Application Laid-Open Nos. 2022-49733 and 2023-19706 using a polyarylate resin for the purpose of improving durability, there is room for improvement in stability of image quality at the time of repeated use.

### SUMMARY OF THE INVENTION

Thus, an object of the present invention is to provide an electrophotographic photosensitive member, a process cartridge, and an electrophotographic apparatus, which have high durability, and also have high stability of image quality.

The above-mentioned object is achieved by the present invention to be described below. That is, according to one aspect of the present invention, there is provided an electrophotographic photosensitive member including: a support; and a photosensitive layer, wherein the photosensitive layer contains a binder resin, a charge generating material, an electron transporting material, and a hole transporting material, wherein the binder resin contains a polyarylate resin having a structural unit represented by the following formula (1), a structural unit represented by the following formula (2), a structural unit represented by the following formula (3), and a structural unit represented by the following formula (4):

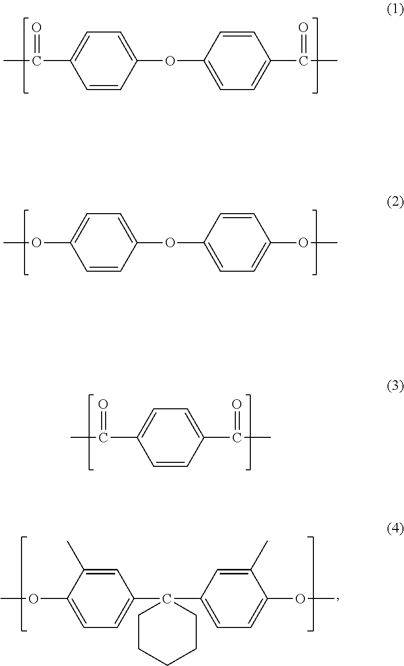

and wherein the hole transporting material contains a compound represented by the following formula (5):

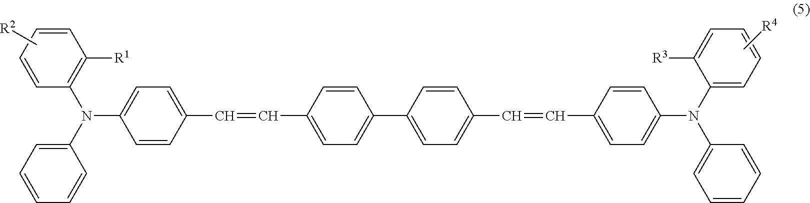

in the formula (5), R¹, R², R³, and R⁴ each independently represent an alkyl group or an alkoxy group having 1 to 4 carbon atoms.

In addition, according to another aspect of the present invention, there is provided a process cartridge including: the above-mentioned electrophotographic photosensitive member; and at least one unit selected from the group consisting of: a charging unit; a developing unit; and a cleaning unit, the process cartridge integrally supporting the electrophotographic photosensitive member and the at least one unit, and being detachably attachable onto a main body of an electrophotographic apparatus.

In addition, according to another aspect of the present invention, there is provided an electrophotographic apparatus including: the above-mentioned electrophotographic photosensitive member; a charging unit; an exposing unit; a developing unit; and a transfer unit.

Further features of the present invention will become apparent from the following description of exemplary embodiments with reference to the attached drawings.

### BRIEF DESCRIPTION OF THE DRAWINGS

FIG. 1 is a view for illustrating an example of a layer configuration of an electrophotographic photosensitive member according to the present invention.

FIG. 2 is a view for illustrating an example of a layer configuration of the electrophotographic photosensitive member according to the present invention.

FIG. 3 is a view for illustrating an example of a layer configuration of the electrophotographic photosensitive member according to the present invention.

FIG. 4 is a view for illustrating an example of a schematic configuration of an electrophotographic apparatus according to the present invention.

FIG. 5 is a view for illustrating an example of a configuration of an image forming unit included in the electrophotographic apparatus according to the present invention.

### DESCRIPTION OF THE EMBODIMENTS

The present invention is described below in detail by way of exemplary embodiments.

In the electrophotographic photosensitive member described in each of Japanese Patent Application Laid-Open Nos. 2022-49733 and 2023-19706, it has been found that image smearing that is one of image defects is liable to occur in a process of repeated use. The inventors have assumed the cause for the foregoing to be as described below. The photosensitive layer containing the polyarylate resin has high wear resistance, and hence a discharge product generated on the surface of the photosensitive member cannot be appropriately removed in a cleaning process.

Based on the above-mentioned assumption, the inventors have made various investigations on a measure for suppressing the occurrence of image smearing in a process of repeated use in a highly durable electrophotographic photosensitive member that uses a polyarylate resin as a binder resin in a photosensitive layer, and as a result, have achieved the present invention.

That is, an electrophotographic photosensitive member according to the present invention includes a support and a photosensitive layer, and the photosensitive layer contains a binder resin, a charge generating material, an electron transporting material, and a hole transporting material. In addition, the binder resin contains a polyarylate resin having a structural unit represented by the following formula (1), a structural unit represented by the following formula (2), a structural unit represented by the following formula (3), and a structural unit represented by the following formula (4).

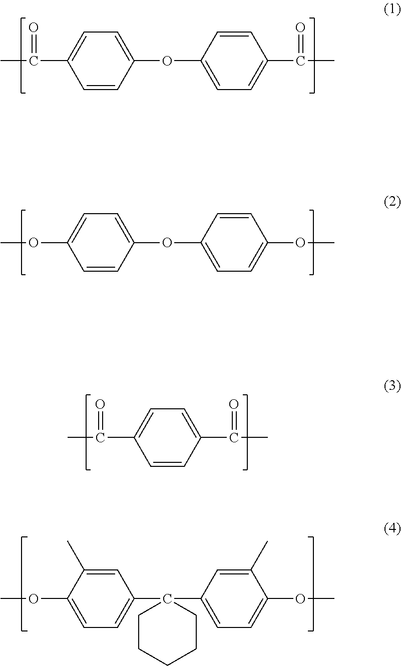

In addition, the hole transporting material contains a compound represented by the following formula (5).

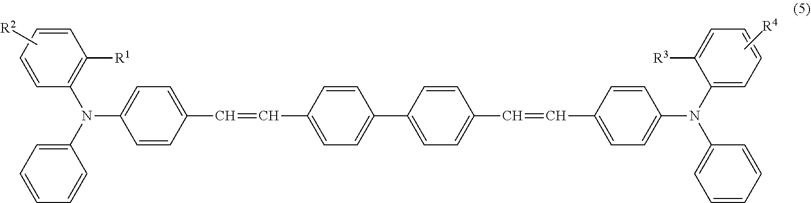

In the formula (5), R¹, R², R³, and R⁴ each independently represent an alkyl group having 1 or more and 4 or less carbon atoms, or an alkoxy group having 1 or more and 4 or less carbon atoms.

The inventors have found that, when the electrophotographic photosensitive member has the above-mentioned configuration according to the present invention, in a process of repeated use, the occurrence of image smearing can be suppressed while high durability is achieved.

The inventors have assumed the reason for the foregoing to be described below. Due to the strong resin interaction, the polyarylate resin having the respective structural units represented by the formulae (1), (2), (3), and (4) has low compatibility with a hole transporting material having a large molecular weight and a rigid skeleton with a high hole transporting ability. However, the hole transporting material represented by the formula (5) has a relatively flexible skeleton while having a large molecular weight, and hence has high compatibility with the polyarylate resin. When the polyarylate resin and the hole transporting material are not satisfactorily mixed with each other due to the low compatibility, concentration unevenness of the hole transporting material occurs on the surface of the photosensitive member, causing micro electrical resistance unevenness. Then, a discharge product is generated by a localized large current caused by discharge unevenness to cause image smearing. Through use of the hole transporting material represented by the formula (5), the electrical resistance unevenness is eliminated and the generation of a discharge product can be suppressed, and hence the occurrence of image smearing can be suppressed.

The configuration of the electrophotographic photosensitive member according to the present invention is described below in more detail.

### [Electrophotographic Photosensitive Member]

The electrophotographic photosensitive member according to the present invention includes a support and a photosensitive layer formed on the support.

A method of producing the electrophotographic photosensitive member according to the present invention is, for example, a method involving: preparing coating liquids for the respective layers to be described later; applying the liquids in a desired order of the layers; and drying the liquids. In this case, examples of the method of applying the coating liquid include dip coating, spray coating, inkjet coating, roll coating, die coating, blade coating, curtain coating, wire bar coating, and ring coating. Of those, dip coating is preferred from the viewpoints of efficiency and productivity.

### (Monolayer Type Photosensitive Member)

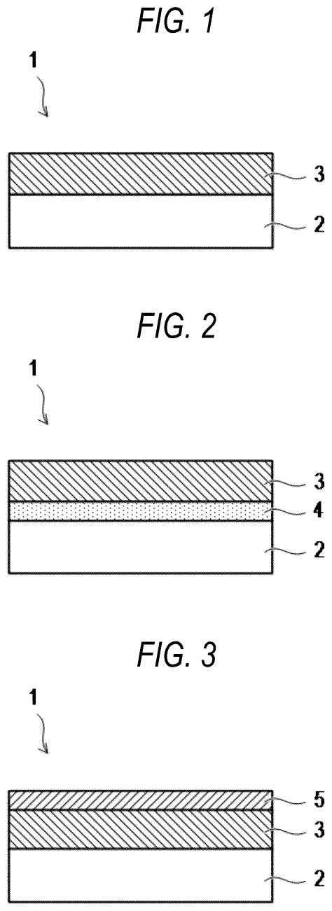

A monolayer type photosensitive member 1 according to one embodiment of the present invention is described below with reference to FIG. 1 to FIG. 3. FIG. 1 to FIG. 3 are each a partial sectional view for illustrating an example of a layer configuration of the monolayer type photosensitive member 1.

As illustrated in FIG. 1, the monolayer type photosensitive member 1 includes, for example, an electroconductive support 2 and a photosensitive layer 3. The photosensitive layer 3 included in the monolayer type photosensitive member 1 is a monolayer type photosensitive layer consisting of a monolayer (one layer). The photosensitive layer 3 contains a binder resin, a charge generating material, an electron transporting material, and a hole transporting material.

As illustrated in FIG. 2, the monolayer type photosensitive member 1 may further include an undercoat layer 4 (intermediate layer) in addition to the support 2 and the photosensitive layer 3. That is, in the monolayer type photosensitive member 1, the photosensitive layer may be formed directly on the support 2 as illustrated in FIG. 1, or may be formed on the support 2 through intermediation of the undercoat layer 4 as illustrated in FIG. 2.

In addition, as illustrated in FIG. 3, the monolayer type photosensitive member 1 may further include a protection layer 5 in addition to the support 2 and the photosensitive layer 3. The protection layer 5 is formed on the photosensitive layer 3. In the present invention, as illustrated in each of FIG. 1 and FIG. 2, it is preferred that the monolayer type photosensitive member 1 do not include the protection layer 5 and the photosensitive layer 3 be formed as a surface layer of the monolayer type photosensitive member 1. When the photosensitive layer 3 containing, as a binder resin, the polyarylate resin (PAR) having the respective structural units represented by the formulae (1) to (4) is formed as a surface layer, the effects of the present invention are easily obtained at the time of repeated use.

The thickness of the photosensitive layer 3 is not particularly limited, but is preferably 5 μm or more and 100 μm or less, more preferably 10 μm or more and 50 μm or less.

### <Support>

In the present invention, the electrophotographic photosensitive member includes the support. In the present invention, the support is preferably an electroconductive support having electroconductivity. In addition, examples of the shape of the support include a cylindrical shape, a belt shape, and a sheet shape. A support having a cylindrical shape out of those shapes is preferred. In addition, the surface of the support may be subjected to, for example, electrochemical treatment such as anodization, blast treatment, or cutting treatment.

A metal, a resin, glass, or the like is preferred as a material for the support.

Examples of the metal include aluminum, iron, nickel, copper, gold, stainless steel, and alloys thereof. An aluminum support using aluminum out of those metals is preferred.

In addition, electroconductivity may be imparted to the resin or the glass through treatment involving, for example, mixing or coating the resin or the glass with an electroconductive material.

### <Undercoat Layer>

In the present invention, an undercoat layer may be arranged on the support. The arrangement of the undercoat layer can improve an adhesive function between layers to impart a charge injection inhibiting function.

The undercoat layer preferably contains a resin. In addition, the undercoat layer may be formed as a cured film by polymerizing a composition containing a monomer having a polymerizable functional group.

Examples of the resin include a polyester resin, a polyarylate resin, a polycarbonate resin, a polyvinyl acetal resin, an acrylic resin, an epoxy resin, a melamine resin, a polyurethane resin, a phenol resin, a polyvinyl phenol resin, an alkyd resin, a polyvinyl alcohol resin, a polyethylene oxide resin, a polypropylene oxide resin, a polyamide resin, a polyamic acid resin, a polyimide resin, a polyamide imide resin, and a cellulose resin.

Examples of the polymerizable functional group of the monomer having the polymerizable functional group include an isocyanate group, a blocked isocyanate group, a methylol group, an alkylated methylol group, an epoxy group, a metal alkoxide group, a hydroxyl group, an amino group, a carboxyl group, a thiol group, a carboxylic acid anhydride group, and a carbon-carbon double bond group.

In addition, the undercoat layer may further contain an electron transporting material, a metal oxide, a metal, an electroconductive polymer, and the like for the purpose of improving electric characteristics. Of those, an electron transporting material and a metal oxide are preferably used.

Examples of the electron transporting material include a quinone compound, an imide compound, a benzimidazole compound, a cyclopentadienylidene compound, a fluorenone compound, a xanthone compound, a benzophenone compound, a cyanovinyl compound, a halogenated aryl compound, a silole compound, and a boron-containing compound. An electron transporting material having a polymerizable functional group may be used as the electron transporting material and copolymerized with the above-mentioned monomer having a polymerizable functional group to form the undercoat layer as a cured film.

Examples of the metal oxide include indium tin oxide, tin oxide, indium oxide, titanium oxide, zinc oxide, aluminum oxide, and silicon dioxide. Examples of the metal include gold, silver, and aluminum.

In addition, the undercoat layer may further contain an additive.

The thickness of the undercoat layer is preferably from 0.1 μm to 50 μm, more preferably from 0.2 μm to 40 μm, particularly preferably from 0.3 μm to 30 μm.

The undercoat layer may be formed by: preparing a coating liquid for an undercoat layer containing the above-mentioned respective materials and a solvent; forming a coating film of the coating liquid; and drying and/or curing the coating film. Examples of the solvent to be used in the coating liquid include an alcohol-based solvent, a ketone-based solvent, an ether-based solvent, an ester-based solvent, and an aromatic hydrocarbon-based solvent.

### <Monolayer Type Photosensitive Layer>

The electrophotographic photosensitive member according to the present invention includes the monolayer type photosensitive layer arranged on the support, or on the undercoat layer arranged on the support.

In the present invention, the monolayer type photosensitive layer contains a binder resin, a charge generating material, a hole transporting material, and an electron transporting material.

### [Binder Resin]

The binder resin to be used in the photosensitive layer contains a polyarylate resin having a structural unit represented by the following formula (1), a structural unit represented by the following formula (2), a structural unit represented by the following formula (3), and a structural unit represented by the following formula (4).

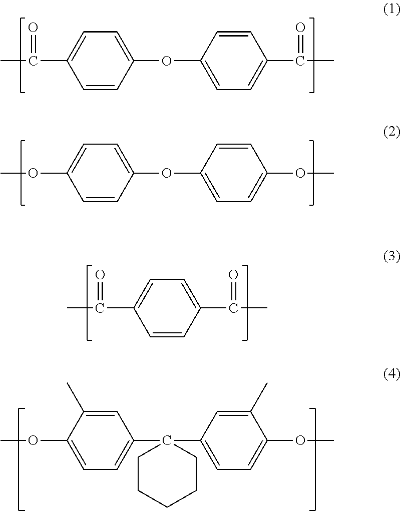

The above-mentioned polyarylate resin may be, for example, a random copolymer, an alternating copolymer, a periodic copolymer, or a block copolymer.

In the above-mentioned polyarylate resin, the ratio of the substance amount in terms of mole of the structural unit represented by the formula (1) to the total substance amount in terms of mole of the structural units for forming the polyarylate resin is represented by M1. In addition, the ratio of the substance amount in terms of mole of the structural unit represented by the formula (2) to the total substance amount in terms of mole of the structural units for forming the polyarylate resin is represented by M2. In addition, the ratio of the substance amount in terms of mole of the structural unit represented by the formula (3) to the total substance amount in terms of mole of the structural units for forming the polyarylate resin is represented by M3. In addition, the ratio of the substance amount in terms of mole of the structural unit represented by the formula (4) to the total substance amount in terms of mole of the structural units for forming the polyarylate resin is represented by M4. In this case, M1/(M1+M3) is preferably more than 0.30, more preferably 0.55 or more from the viewpoint of improving wear resistance.

In addition, M2/(M2+M4) is preferably larger from the viewpoint of improving wear resistance, and specifically, is preferably 0.10 or more. In addition, M2/(M2+M4) is preferably less than 0.50 from the viewpoint of solubility in a solvent. When the solubility in a solvent is improved, a photosensitive layer can be satisfactorily formed. Thus, M2/(M2+M4) is preferably 0.10 or more and less than 0.50.

In addition, M3/(M1+M3) is preferably 0.30 or more and less than 0.70 from the viewpoint of suppressing an increase in residual potential.

In addition, M4/(M2+M4) is preferably less than 0.90 from the viewpoint of improving solubility in a solvent.

In the above-mentioned polyarylate resin, the sum of the ratio of the substance amount in terms of mole of the structural unit represented by the formula (1) and the ratio of the substance amount in terms of mole of the structural unit represented by the formula (3) is preferably 0.50 or more with respect to the total substance amount in terms of mole of structural units derived from dicarboxylic acids for forming the polyarylate resin.

In the above-mentioned polyarylate resin, the sum of the ratio of the substance amount in terms of mole of the structural unit represented by the formula (2) and the ratio of the substance amount in terms of mole of the structural unit represented by the formula (4) is preferably more than 0 and 0.50 or less with respect to the total substance amount in terms of mole of structural units derived from bisphenols for forming the polyarylate resin.

In addition, the ratio of the substance amount in terms of mole of the structural unit represented by the formula (1) is preferably 0.30 or more, more preferably 0.50 or more with respect to the total substance amount in terms of mole of structural units derived from dicarboxylic acids for forming the above-mentioned polyarylate resin.

In addition, in the electrophotographic photosensitive member according to the present invention, the ratio of the mass of the above-mentioned polyarylate resin to the total mass of the binder resin is preferably 50 mass % or more.

The viscosity-average molecular weight of the above-mentioned polyarylate resin (PAR) is preferably 10,000 or more, more preferably 30,000 or more, still more preferably 50,000 or more. When the viscosity-average molecular weight of the above-mentioned polyarylate resin (PAR) is 10,000 or more, the wear resistance of the photosensitive member is improved. Meanwhile, the viscosity-average molecular weight of the above-mentioned polyarylate resin (PAR) is preferably 80,000 or less, more preferably 70,000 or less. When the viscosity-average molecular weight of the above-mentioned polyarylate resin (PAR) is 80,000 or less, the above-mentioned polyarylate resin (PAR) is easily dissolved in a solvent for forming a photosensitive layer.

Examples of the bisphenols for forming bisphenol-derived repeating units in the above-mentioned polyarylate resin include a compound represented by the following formula (BP-1) and a compound represented by the following formula (BP-2). The compound represented by the following formula (BP-1) is hereinafter sometimes referred to as “compound (BP-1).” In addition, the compound represented by the following formula (BP-2) is hereinafter sometimes referred to as “compound (BP-2).”

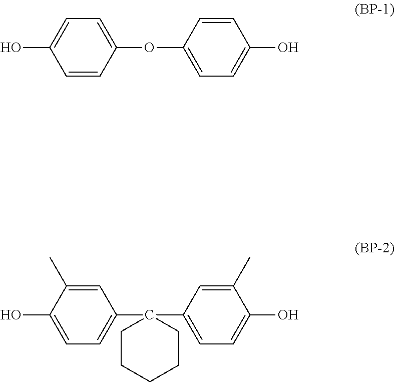 [SMILES: Oc1ccc(Oc2ccc(O)cc2)cc1 ; Cc1cc(C2(c3ccc(O)c(C)c3)CCCCC2)ccc1O]

In addition, examples of the dicarboxylic acids for forming dicarboxylic acid-derived repeating units in the above-mentioned polyarylate resin include a compound represented by the following formula (DC-1) and a compound represented by the following formula (DC-2). The compound represented by the following formula (DC-1) is hereinafter sometimes referred to as “compound (DC-1).” In addition, the compound represented by the following formula (DC-2) is hereinafter sometimes referred to as “compound (DC-2).”

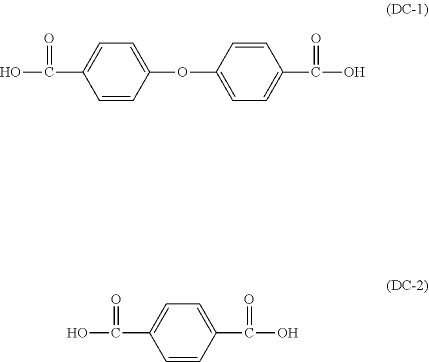 [SMILES: O=C(O)c1ccc(Oc2ccc(C(=O)O)cc2)cc1 ; O=C(O)c1ccc(C(=O)O)cc1]

A bisphenol ratio in the resin may be adjusted by changing the amounts of the compound (BP-1) and the compound (BP-2) to be added at the time of production of the above-mentioned polyarylate resin (PAR). In addition, a dicarboxylic acid ratio in the resin may be similarly adjusted by changing the amounts of the compound (DC-1) and the compound (DC-2) to be added at the time of production of the above-mentioned polyarylate resin (PAR).

The bisphenols (e.g., the compounds (BP-1) and (BP-2)) may each be used by being derivatized into an aromatic diacetate. The dicarboxylic acids (e.g., the compounds (DC-1) and (DC-2)) may each be used by being derivatized. Examples of the derivative of the dicarboxylic acid include a dicarboxylic acid dichloride, a dicarboxylic acid dimethyl ester, a dicarboxylic acid diethyl ester, and a dicarboxylic acid anhydride. The dicarboxylic acid dichloride is a compound having a structure in which two “—C(═O)—OH” groups of the dicarboxylic acid are each substituted with a “—C(═O)—Cl” group.

In the polycondensation of the bisphenol and the dicarboxylic acid, one or both of a base and a catalyst may be added. An example of the base is sodium hydroxide. Examples of the catalyst include benzyltributylammonium chloride, ammonium chloride, ammonium bromide, a quaternary ammonium salt, triethylamine, and trimethylamine.

The photosensitive layer may contain, as the binder resin, only the above-mentioned polyarylate resin (PAR), and may further contain a binder resin other than the foregoing (hereinafter sometimes referred to as “other binder resin”).

Examples of the other binder resin include: thermoplastic resins (more specifically, a polyarylate resin other than the above-mentioned polyarylate resin (PAR), a polycarbonate resin, a styrene-based resin, a styrene-butadiene copolymer, a styrene-acrylonitrile copolymer, a styrene-maleic acid copolymer, a styrene-acrylic acid copolymer, an acrylic copolymer, a polyethylene resin, an ethylene-vinyl acetate copolymer, a chlorinated polyethylene resin, a polyvinyl chloride resin, a polypropylene resin, an ionomer, a vinyl chloride-vinyl acetate copolymer, a polyester resin, an alkyd resin, a polyamide resin, a polyurethane resin, a polysulfone resin, a diallyl phthalate resin, a ketone resin, a polyvinyl butyral resin, a polyvinyl acetal resin, and a polyether resin); thermosetting resins (more specifically, a silicone resin, an epoxy resin, a phenol resin, a urea resin, a melamine resin, and any other crosslinkable thermosetting resin); and photocurable resins (more specifically, an epoxy-acrylic acid-based resin and a urethane-acrylic acid-based copolymer).

The structure of the polyarylate resin of the present invention may be determined by a ¹H-nuclear magnetic resonance spectrum obtained by performing component analysis of polymer components recovered from the photosensitive layer through use of ¹H-nuclear magnetic resonance spectrometry in deuterated chloroform.

An example of a specific analysis method for the polyarylate resin in the photosensitive layer when the photosensitive member is a cylindrical body is described below.

### (Reprecipitation of Resin in Photosensitive Layer)

The photosensitive member is cut at a position 10 cm distant from an end portion of the photosensitive member in a generating line direction with a scroll saw.
An inner surface of the cut cylindrical body of 10 cm is wiped with lens cleaning paper impregnated with chloroform.
To elute the photosensitive layer, 3 cm of an end portion of the cut cylindrical body is immersed in chloroform. (About 60 ml of chloroform is loaded into a 100 mL beaker, and the end portion is immersed therein at normal temperature for 5 minutes.)
The chloroform solution in which the above-mentioned photosensitive layer has been eluted is concentrated to 2 mL with a rotary evaporator, and the concentration is stopped.
50 mL of a methanol/acetone mixed solution (volume ratio: 1:1) is prepared, and the whole amount of the above-mentioned concentrated solution is dropped thereinto while the mixed solution is stirred, to thereby perform reprecipitation.
Suction filtration is performed with a funnel (funnel: SU-40, paper filter: No. 5C-40, manufactured by Kiriyama Glass Co.).
The residue on the paper filter is recovered with a spatula and dried in a vacuum (70° C., 1 hour).

### (NMR Measurement)

To prepare a measurement sample, 20 mg of a sample is dissolved in 1 g of deuterated chloroform containing tetramethylsilane serving as a reference material, and the whole amount thereof is transferred to an NMR tube.
(Deuterated chloroform: manufactured by Sigma-Aldrich Japan G.K., chloroform-d, model number: 612200)
(NMR tube: manufactured by Norell, Inc., ST500-7, model number: S3010)
NMR measurement is performed.
   Apparatus: AVANCE 500 manufactured by Bruker
   Conditions: Proton NMR, automatic measurement by Icon-NMR
   Number of scans: 32
   Reference peak: The peak of a methyl group of tetramethylsilane is set to 0 ppm.

### [Hole Transporting Material]

The hole transporting material contains a compound represented by the following formula (5).

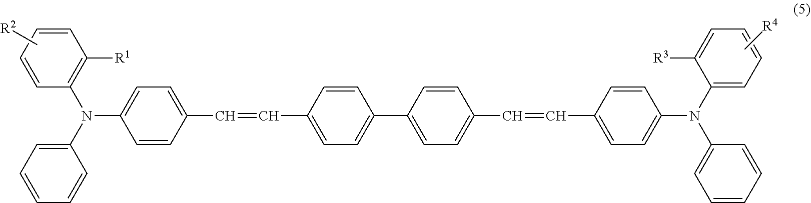

In the formula (5), R¹, R², R³ and R⁴ each independently represent an alkyl group having 1 or more and 4 or less carbon atoms, or an alkoxy group having 1 or more and 4 or less carbon atoms.

It is preferred that, in the formula (5), substitution positions of R² and R⁴ be each a 6-position. In addition, it is preferred that, in the formula (5), R¹ and R³ represent groups identical to each other, and R² and R⁴ represent groups identical to each other.

Examples of the alkyl group represented by each of R¹, R², R³ and R⁴ include alkyl groups each having 1 or more and 4 or less carbon atoms, such as a methyl group, an ethyl group, a n-propyl group, an isopropyl group, a n-butyl group, an isobutyl group, a s-butyl group, and a t-butyl group.

In the compound represented by the formula (5), a cis-isomer and a trans-isomer are present. Of those, a trans-isomer is preferably used.

Suitable examples of the compound represented by the formula (5) include compounds shown in Table 1. Examples of the compound represented by the formula (5) shown in Table 1 are hereinafter sometimes referred to as “hole transporting materials (H-1) to (H-10),” respectively. The hole transporting materials (H-1) to (H-10) are each a trans-isomer.

Regarding the compound represented by the formula (5), a ¹H-NMR spectrum may be obtained by ¹H-nuclear magnetic resonance spectrometry in a solvent such as deuterated chloroform. As a result, the structure represented by the formula (5) can be identified.

TABLE 1

| Compound represented | | | | |
|---|---|---|---|---|
| by formula (5) | R¹ | R² | R³ | R⁴ |
| H-1 | Me | 6-Et | Me | 6-Et |
| H-2 | Et | 6-Et | Et | 6-Et |
| H-3 | i-Pr | 6-i-Pr | i-Pr | 6-i-Pr |
| H-4 | Me | 6-t-Bu | Me | 6-t-Bu |
| H-5 | Me | 6-OMe | Me | 6-OMe |
| H-6 | OMe | 6-OMe | OMe | 6-OMe |
| H-7 | Me | 4-OMe | Me | 4-OMe |
| H-8 | OEt | 6-OEt | OEt | 6-OMe |
| H-9 | Me | 6-Et | Et | 6-Et |
| H-10 | Me | 6-Et | i-Pr | 6-i-Pr |

In Table 1, Me represents a methyl group, Et represents an ethyl group, i-Pr represents an isopropyl group, t-Bu represents a t-butyl group, OMe represents a methoxy group, and OEt represents an ethoxy group.

In addition, in Table 1, the numbers in the structures of R² and R⁴ indicate the substitution positions of R² and R⁴ in formula (5).

The content of the hole transporting material in the photosensitive layer is preferably 10 parts by mass or more and 200 parts by mass or less, more preferably 30 parts by mass or more and 120 parts by mass or less, still more preferably 50 parts by mass or more and 90 parts by mass or less with respect to 100 parts by mass of the binder resin.

The photosensitive layer may further contain one or more kinds of hole transporting materials other than the compound represented by the formula (5) (hereinafter sometimes referred to as “other hole transporting materials”). Examples of the other hole transporting material include a triphenylamine derivative, a diamine derivative (e.g., an N,N,N′,N′-tetraphenylbenzidine derivative, an N,N,N′,N′-tetraphenylphenylenediamine derivative, an N,N,N′,N′-tetraphenylnaphthylenediamine derivative, an N,N,N′,N′-tetraphenylphenanthrenediamine derivative, and a di(aminophenylethenyl)benzene derivative), an oxadiazole-based compound (e.g., 2,5-di(4-methylaminophenyl)-1,3,4-oxadiazole), a styryl-based compound (e.g., 9-(4-diethylaminostyryl)anthracene), a carbazole-based compound (e.g., polyvinyl carbazole), an organic polysilane compound, a pyrazoline-based compound (e.g., 1-phenyl-3-(p-dimethylaminophenyl)pyrazoline), a hydrazone-based compound, an indole-based compound, an oxazole-based compound, an isooxazole-based compound, a thiazole-based compound, a thiadiazole-based compound, an imidazole-based compound, a pyrazole-based compound, and a triazole-based compound.

It is preferred that the ratio of the substance amount in terms of mole of the compound represented by the formula (5) in the photosensitive layer to the total substance amount in terms of mole of all of the hole transporting materials in the photosensitive layer be 0.6 or more.

It is preferred that the ratio of the compound represented by the formula (5) in the photosensitive layer to the content of all of the hole transporting materials in the photosensitive layer be 50 mass % or more.

### [Charge Generating Material]

Examples of the charge generating material include a phthalocyanine-based pigment, a perylene-based pigment, a bisazo pigment, a trisazo pigment, a dithioketopyrrolopyrrole pigment, a metal-free naphthalocyanine pigment, a metal naphthalocyanine pigment, a squaraine pigment, an indigo pigment, an azulenium pigment, a cyanine pigment, powder of an inorganic photoconductive material (e.g., selenium, selenium-tellurium, selenium-arsenic, cadmium sulfide, and amorphous silicon), a pyrylium pigment, an anthanthrone-based pigment, a triphenylmethane-based pigment, a threne-based pigment, a toluidine-based pigment, a pyrazoline-based pigment, and a quinacridone-based pigment. The photosensitive layer may contain only one kind of charge generating material, or may contain two or more kinds of charge generating materials.

The phthalocyanine-based pigment is a pigment having a phthalocyanine structure. Examples of the phthalocyanine-based pigment include metal-free phthalocyanine and a metal phthalocyanine. Examples of the metal phthalocyanine include titanyl phthalocyanine, hydroxygallium phthalocyanine, and chlorogallium phthalocyanine. The metal phthalocyanine is preferably titanyl phthalocyanine. Titanyl phthalocyanine is represented by the following formula (CGM-1).

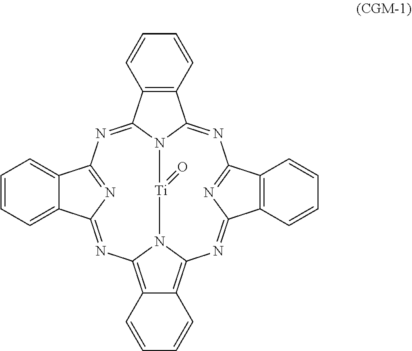

The phthalocyanine-based pigment may be crystalline or amorphous. An example of the crystal of metal-free phthalocyanine is an X-form crystal of metal-free phthalocyanine (hereinafter sometimes referred to as “X-form metal-free phthalocyanine”). Examples of the crystal of titanyl phthalocyanine include α-form, β-form, and Y-form crystals of titanyl phthalocyanine (hereinafter sometimes referred to as “α-form, β-form, and Y-form titanyl phthalocyanines,” respectively).

For example, in a digital optical image forming apparatus (e.g., a laser beam printer or a facsimile using a light source such as a semiconductor laser), a photosensitive member having sensitivity in a wavelength region of 700 nm or more is preferably used. The charge generating material is preferably a phthalocyanine-based pigment, more preferably metal-free phthalocyanine or titanyl phthalocyanine, because these materials each have a high quantum yield in a wavelength region of 700 nm or more. In addition, the charge generating material is still more preferably titanyl phthalocyanine, particularly preferably Y-form titanyl phthalocyanine.

The Y-form titanyl phthalocyanine does not have a peak at 26.2°, and has a main peak, for example, at a Bragg angle (2θ+0.2°) of 27.2° in a CuKα characteristic X-ray diffraction spectrum. The main peak in the CuKα characteristic X-ray diffraction spectrum is a peak having the first or second largest intensity in the range of a Bragg angle (2θ±0.2°) of 3° or more and 400 or less.

The CuKα characteristic X-ray diffraction spectrum may be measured, for example, by the following method. First, a sample (titanyl phthalocyanine) is loaded into a sample holder of an X-ray diffraction apparatus (e.g., “RINT (trademark) 1100” manufactured by Rigaku Corporation). Subsequently, an X-ray diffraction spectrum is measured under the conditions of an X-ray tube bulb of Cu, a tube voltage of 40 kV, a tube current of 30 mA, and a CuKα characteristic X-ray wavelength of 1.542 Å. A measurement range (2θ) is, for example, 3° or more and 400 or less (start angle: 3° and stop angle: 40°), and a scanning speed is, for example, 10°/min. The main peak is determined from the resultant X-ray diffraction spectrum, and the Bragg angle of the main peak is read.

The content of the charge generating material in the photosensitive layer is preferably 0.1 part by mass or more and 50 parts by mass or less, more preferably 0.5 part by mass or more and 5 parts by mass or less with respect to 100 parts by mass of the binder resin.

### [Electron Transporting Material]

It is preferred that the electron transporting material contain at least one compound selected from the group consisting of: a compound represented by the following formula (6); a compound represented by the following formula (7); a compound represented by the following formula (8); a compound represented by the following formula (9); a compound represented by the following formula (10); a compound represented by the following formula (11); and a compound represented by the following formula (12). It is conceived that, when the photosensitive layer contains the above-mentioned electron transporting material, the compatibility between the above-mentioned polyarylate resin and the compound represented by the formula (5) is increased to enhance the uniformity in the photosensitive layer, and thus the effects of the present invention are highly achieved.

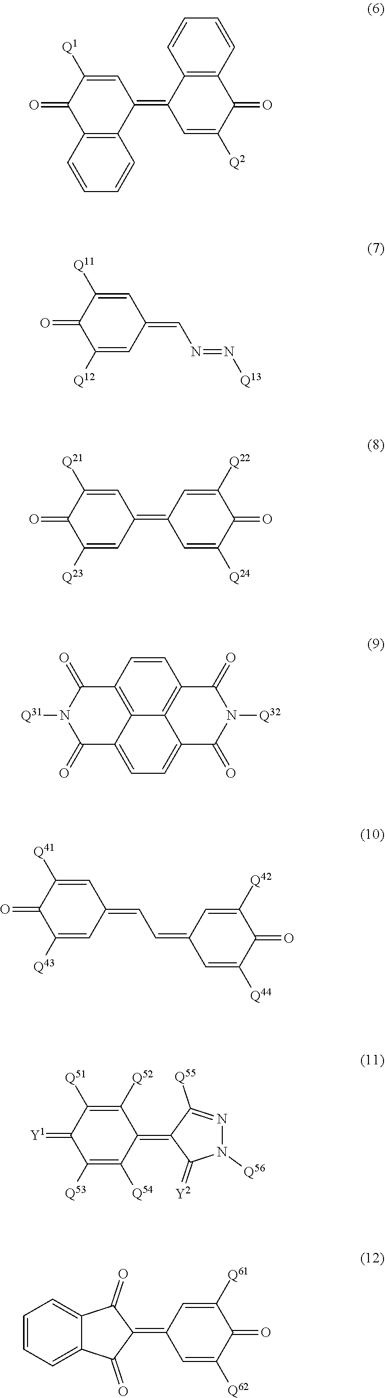

Q¹ and Q² in the formula (6), Q¹¹, Q¹², and Q¹³ in the formula (7), Q²¹, Q²², Q²³, and Q²⁴ in the formula (8), Q³¹ and Q³² in the formula (9), Q⁴¹, Q⁴², Q⁴³, and Q⁴⁴ in the formula (10), Q⁵¹, Q⁵², Q⁵³, Q⁵⁴, Q⁵⁵, and Q⁵⁶ in the formula (11), and Q⁶¹ and Q⁶² in the formula (12) each independently represent a hydrogen atom, a halogen atom, a cyano group, an alkyl group having 1 or more and 6 or less carbon atoms, an alkenyl group having 2 or more and 6 or less carbon atoms, an alkoxy group having 1 or more and 6 or less carbon atoms, or an aryl group having 6 or more and 14 or less carbon atoms that may be substituted with at least one substituent selected from the group consisting of: an alkyl group having 1 or more and 6 or less carbon atoms; and a halogen atom. Y¹ and Y² in the formula (11) each independently represent an oxygen atom or a sulfur atom.

It is preferred that Q¹ and Q² in the formula (6), Q¹¹ to Q¹³ in the formula (7), Q²¹ to Q²⁴ in the formula (8), Q³¹ and Q³² in the formula (9), Q⁴¹ to Q⁴⁴ in the formula (10), Q⁵¹ to Q⁵⁶ in the formula (11), and Q⁶¹ and Q⁶² in the formula (12) each independently represent a hydrogen atom, an alkyl group having 1 or more and 6 or less carbon atoms, or an aryl group having 6 or more and 14 or less carbon atoms that may be substituted with at least one substituent selected from the group consisting of: an alkyl group having 1 or more and 6 or less carbon atoms; and a halogen atom. It is preferred that Y¹ and Y² in the formula (11) each represent an oxygen atom.

The alkyl group having 1 or more and 6 or less carbon atoms, which is represented by each of Q¹ and Q² in the formula (6), Q¹¹ to Q¹³ in the formula (7), Q²¹ to Q²⁴ in the formula (8), Q³¹ and Q³² in the formula (9), Q⁴¹ to Q⁴⁴ in the formula (10), Q⁵¹ to Q⁵⁶ in the formula (11), and Q⁶¹ and Q⁶² in the formula (12), is preferably an alkyl group having 1 or more and 5 or less carbon atoms, preferably a methyl group, an ethyl group, a propyl group, a butyl group, or a pentyl group, particularly preferably a methyl group, an isopropyl group, a tert-butyl group, or a 1,1-dimethylpropyl group.

The aryl group having 6 or more and 14 or less carbon atoms, which is represented by each of Q¹ and Q² in the formula (6), Q¹¹ to Q¹³ in the formula (7), Q²¹ to Q²⁴ in the formula (8), Q³¹ and Q³² in the formula (9), Q⁴¹ to Q⁴⁴ in the formula (10), Q⁵¹ to Q⁵⁶ in the formula (11), and Q⁶¹ and Q⁶² in the formula (12), is preferably an aryl group having 6 or more and 10 or less carbon atoms, more preferably a phenyl group.

Here, the alkyl group having 1 or more and 6 or less carbon atoms that the aryl group having 6 or more and 14 or less carbon atoms may have as a substituent is preferably an alkyl group having 1 or more and 3 or less carbon atoms, more preferably a methyl group or an ethyl group.

In addition, the halogen atom that the aryl group having 6 or more and 14 or less carbon atoms may have as a substituent is preferably a fluorine atom, a chlorine atom, or a bromine atom, particularly preferably a chlorine atom.

When the aryl group having 6 or more and 14 or less carbon atoms is substituted with a substituent, the number of substituents is preferably 1 or more and 5 or less, more preferably 1 or 2.

The aryl group having 6 or more and 14 or less carbon atoms that is substituted with at least one substituent selected from the group consisting of: an alkyl group having 1 or more and 6 or less carbon atoms; and a halogen atom is preferably a chlorophenyl group, a dichlorophenyl group, or an ethylmethylphenyl group, more preferably a 4-chlorophenyl group, a 2,5-dichlorophenyl group, or a 2-ethyl-6-methylphenyl group.

A suitable example of the compound represented by the formula (6) is a compound represented by the following formula (E-4). A suitable example of the compound represented by the formula (7) is a compound represented by the following formula (E-5). A suitable example of the compound represented by the formula (8) is a compound represented by the following formula (E-7). A suitable example of the compound represented by the formula (9) is a compound represented by the following formula (E-6). A suitable example of the compound represented by the formula (10) is a compound represented by the following formula (E-8). Suitable examples of the compound represented by the formula (11) include a compound represented by the following formula (E-2) and a compound represented by the following formula (E-3). A suitable example of the compound represented by the formula (12) is a compound represented by the following formula (E-1). The compounds represented by the formulae (E-1) to (E-8) are hereinafter sometimes referred to as “electron transporting materials (E-1) to (E-8),” respectively.

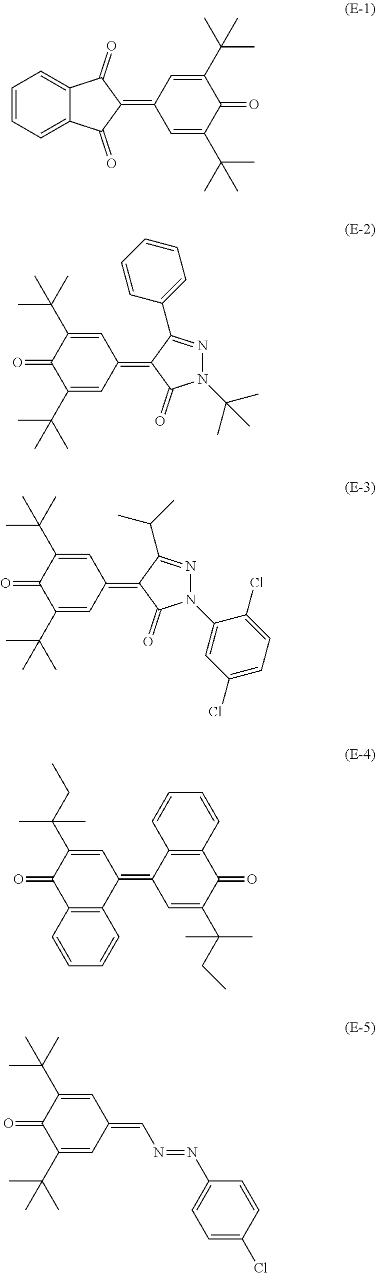 [SMILES: CC(C)(C)C1=CC(=C2C(=O)c3ccccc3C2=O)C=C(C(C)(C)C)C1=O ; CC(C)(C)C1=CC(=C2C(=O)N(C(C)(C)C)N=C2c2ccccc2)C=C(C(C)(C)C)C1=O ; CC(C)C1=NN(c2cc(Cl)ccc2Cl)C(=O)C1=C1C=C(C(C)(C)C)C(=O)C(C(C)(C)C)=C1 ; CCC(C)(C)C1=C/C(=C2/C=C(C(C)(C)CC)C(=O)c3ccccc32)c2ccccc2C1=O ; CC(C)(C)C1=CC(=C/N=N/c2ccc(Cl)cc2)C=C(C(C)(C)C)C1=O]

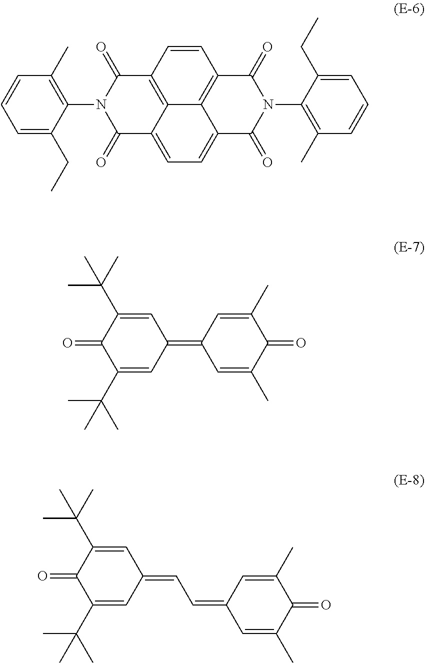 [SMILES: CCc1cccc(C)c1N1C(=O)c2ccc3c4c(ccc(c24)C1=O)C(=O)N(c1c(C)cccc1CC)C3=O ; CC1=CC(=C2C=C(C(C)(C)C)C(=O)C(C(C)(C)C)=C2)C=C(C)C1=O ; CC1=CC(=CC=C2C=C(C(C)(C)C)C(=O)C(C(C)(C)C)=C2)C=C(C)C1=O]

The content of the electron transporting material in the photosensitive layer is preferably 5 parts by mass or more and 150 parts by mass or less, more preferably 10 parts by mass or more and 100 parts by mass or less, still more preferably 30 parts by mass or more and 70 parts by mass or less with respect to 100 parts by mass of the binder resin. The photosensitive layer may contain only one kind of electron transporting material, or may contain two or more kinds of electron transporting materials.

### [Additive]

The photosensitive layer may contain an additive as required. Examples of the additive include a UV absorber, an antioxidant, a radical scavenger, a singlet quencher, a softener, a surface modifier, an extender, a thickener, a wax, a donor, a surfactant, a plasticizer, a sensitizer, and a leveling agent. In particular, it is preferred that the photosensitive layer contain at least one compound selected from the group consisting of: a compound represented by the following formula (T-1); a compound represented by the following formula (T-2); a compound represented by the following formula (T-3); and a compound represented by the following formula (T-4). “t-Bu” in each of the formulae (T-2), (T-3), and (T-4) represents a tert-butyl group.

The content ratio of the additive in the photosensitive layer is preferably 0.1 mass % or more and 10 mass % or less with respect to the total mass of the photosensitive layer.

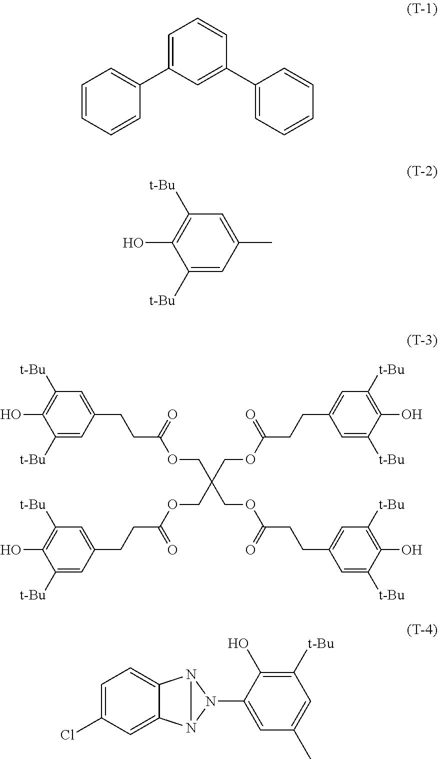

Subsequently, a process cartridge and an electrophotographic apparatus according to the present invention are described. The process cartridge according to the present invention is characterized by integrally supporting the electrographic photosensitive member described above, and at least one unit selected from the group consisting of: a charging unit; a developing unit; and a cleaning unit, and being detachably attachable onto a main body of an electrophotographic apparatus.

In addition, the electrophotographic apparatus according to the present invention is characterized by including the electrophotographic photosensitive member described above, and a charging unit, an exposing unit, a developing unit, and a transfer unit.

The charging unit can be a charging unit configured to positively charge the electrophotographic photosensitive member.

### [Electrophotographic Apparatus]

An example of a configuration of the electrophotographic apparatus including the process cartridge according to the present invention is described below in detail.

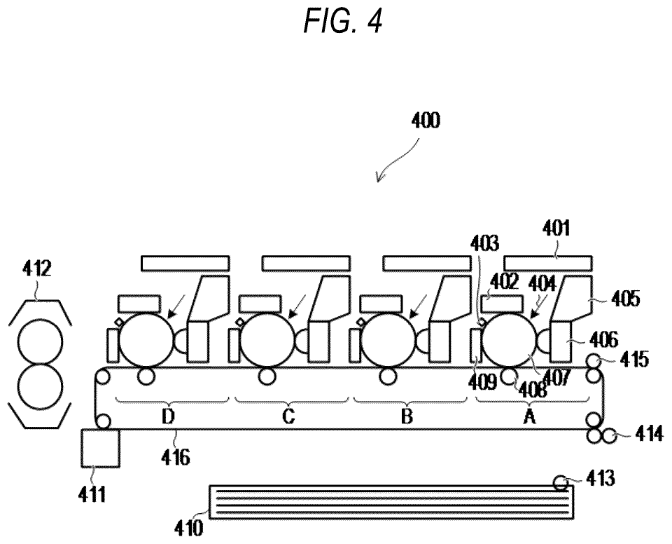

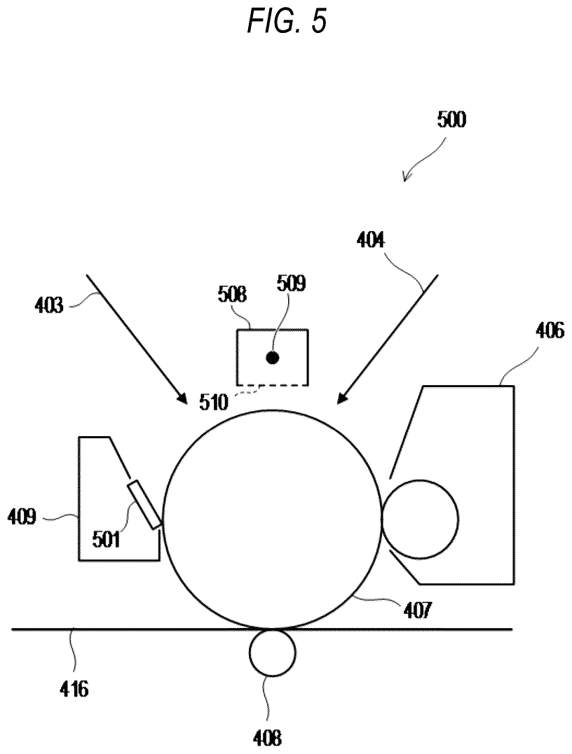

FIG. 4 is a schematic view for illustrating an example of a configuration of the electrophotographic apparatus according to the present invention. FIG. 5 is a schematic view for illustrating an example of a configuration of an image forming unit included in the electrophotographic apparatus illustrated in FIG. 4.

In an electrophotographic apparatus 400, image forming units A, B, C, and D each including a photosensitive member 407 are arranged in series as illustrated in FIG. 4. The image forming units A, B, C, and D each include the photosensitive member 407, a charging device 402, and a developing device 406, and the electrophotographic apparatus 400 is configured to form an image by superimposing toner images formed on the respective photosensitive members 407 on a transfer material such as paper.

Toners to be used in the respective image forming units A, B, C, and D are toners corresponding to yellow, magenta, cyan, and black colors, respectively, and toner images formed by the respective image forming units are superimposed to form a full-color image.

The transfer material is picked up from a transfer material cassette 410 by a pickup roller 413, conveyed by a conveyance roller 414, adjusted for position by a registration roller 415, and adsorbed onto a transfer material conveyance and transfer belt 416. After that, images sequentially formed by the respective image forming units A, B, C, and D are transferred onto the transfer material, and the toner images transferred onto the transfer material are each fixed by a fixing device 412. In addition, toner, paper dust, and the like on the transfer material conveyance and transfer belt 416 are cleaned by a belt cleaning device 411.

Each of the image forming units A, B, C, and D includes the photosensitive member 407, the charging device 402, an exposing device 401, the developing device 406, and a transfer device 408. In each of the image forming units A, B, C, and D, the charging device 402 charges the photosensitive member 407, and the exposing device 401 exposes the charged photosensitive member 407 to exposure light 404 to form an electrostatic latent image. In addition, the developing device 406 develops the electrostatic latent image with toner, and the transfer device 408 transfers the toner image onto the transfer material.

A toner replenishing device 405 is arranged on the developing device 406. Further, a cleaning device 409 that removes residual toner from the surface of the photosensitive member 407 after the transfer, and a pre-exposing device 403 are arranged on the periphery of the photosensitive member 407.

A process cartridge 500 configured to be detachably attachable onto a main body of the electrophotographic apparatus 400 integrally supports the photosensitive member 407, the charging device 402, the developing device 406, and the cleaning device 409.

The transfer device 408 preferably includes a contact transfer unit. A belt, a roller, or a drum is preferably used as the contact transfer unit.

In FIG. 4, there is illustrated an example of a configuration in which an image is transferred directly onto the transfer material conveyed by the transfer material conveyance and transfer belt 416 through use of the transfer device 408, but a configuration in which an image is first transferred onto an intermediate transfer member and then transferred from the intermediate transfer member onto the transfer material may be adopted.

In the cleaning device 409, blade cleaning, roller cleaning, fur brush cleaning, mag roller cleaning, or the like is used. In particular, as illustrated in FIG. 5, blade cleaning is suitably used. In the example illustrated in FIG. 5, a cleaning blade 501 is used, and toner that is scraped off is stored. From the viewpoint of preventing the surface of the photosensitive member from being scratched, a cleaner-less configuration is preferred.

As the exposing device 401 for forming an electrostatic latent image, a known unit, such as a device that emits laser light as the exposure light 404 or a device including a light emitting diode (LED) as a light source, is used.

The photosensitive member 407 is the electrophotographic photosensitive member according to the present invention described above.

The charging device 402 includes a corona charging member 508. The corona charging member 508 charges the photosensitive member 407 without being brought into contact with the photosensitive member 407 as illustrated in detail in FIG. 5.

The corona charging member 508 includes a charge control unit 510 that controls a charge potential and a discharge electrode 509.

A metal wire having a diameter of preferably from 10 μm to 500 μm, more preferably from 50 μm to 200 μm is preferably used as the discharge electrode 509 of the corona charging member 508. Tungsten, stainless steel, or the like coated with a precious metal such as gold as required is used as a material for the metal wire. In addition, a needle-like discharge electrode may be used as the discharge electrode 509.

When the metal wire or the needle-like electrode is used as the discharge electrode 509, the charge control unit 510 is arranged between the discharge electrode 509 and the photosensitive member 407. When the charge control unit 510 is arranged, the charge potential of the photosensitive member 407 can be set to a desired potential.

In a scorotron charger, a grid wire having a voltage applied thereto or the like is used as the charge control unit 510. In addition, a unit having a configuration in which a metal thin plate of, for example, stainless steel (SUS) is etched to allow the passage of an ion flow may be preferably used as the charge control unit 510 instead of the grid wire.

Meanwhile, in the case of a corotron charger, the charge potential is controlled by the amount of current, but the charge potential obtained by the corotron charger has environmental dependency. In view of the foregoing, when high image quality is pursued, the charge potential cannot be sufficiently controlled in the corotron charger in some cases, and hence the scorotron charger is preferably used.

The example in which the electrophotographic apparatus 400 is a full-color printer is illustrated in FIG. 4, but the electrophotographic apparatus according to the present invention is not limited thereto. For example, the electrophotographic apparatus according to the present invention may be a monochrome printer. In addition, the electrophotographic apparatus according to the present invention is not limited to a printer, and may be, for example, a copying machine or a multifunction peripheral.

According to the present invention, an electrophotographic photosensitive member, a process cartridge, and an electrophotographic apparatus, which have high durability, and have high stability of image quality at the time of repeated use, can be provided.

### EXAMPLES

The present invention is described below in more detail by way of Examples and Comparative Examples. The present invention is by no means limited by the following Examples within a scope not departing from the gist of the present invention. In the following description of Examples, the term “part(s)” is by mass unless otherwise specified.

### <Production of Polyarylate Resin (PAR)>

### Synthesis of Resin (PAR-1)

A three-necked flask including a temperature gauge, a three-way cock, and a dropping funnel was used as a reaction vessel.

First, the following materials were prepared.
Compound (BP-2) (28.7 mmol) serving as a monomer
Compound (BP-1) (12.3 mmol) serving as a monomer
2,6-Dimethylphenol (DMP) (0.413 mmol) serving as an end terminator
Sodium hydroxide (98 mmol)
Benzyltributylammonium chloride (0.384 mmol)

The above-mentioned materials were loaded into the reaction vessel, and air in the reaction vessel was replaced by an argon gas. Water (300 mL) was added to the contents of the reaction vessel. The contents of the reaction vessel were stirred at 50° C. for 1 hour. The contents of the reaction vessel were cooled to 10° C. to provide an alkaline aqueous solution A.

Next, a dicarboxylic acid dichloride (20.8 mmol) of the compound (DC-1) serving as a monomer, and a dicarboxylic acid dichloride (11.2 mmol) of the compound (DC-2) serving as a monomer were dissolved in chloroform (150 mL). As a result, a chloroform solution B was obtained.

The chloroform solution B was slowly dropped into the alkaline aqueous solution A over 110 minutes through use of the dropping funnel. The contents of the reaction vessel were stirred for 4 hours to allow a polymerization reaction to proceed while the temperature (liquid temperature) of the contents of the reaction vessel was regulated to 15±5° C. The upper layer (aqueous layer) of the contents of the reaction vessel was removed by decantation. Thus, an organic layer was obtained. Next, ion-exchanged water (400 mL) was loaded into an Erlenmeyer flask. The resultant organic layer was further added to the Erlenmeyer flask. Chloroform (400 mL) and acetic acid (2 mL) were further added to the Erlenmeyer flask. The contents of the Erlenmeyer flask were stirred at room temperature (25° C.) for 30 minutes. The upper layer (aqueous layer) of the contents of the Erlenmeyer flask was removed by decantation. Thus, an organic layer was obtained. The resultant organic layer was washed with ion-exchanged water (1 L) through use of a separating funnel. The washing with ion-exchanged water was repeated five times. Thus, a water-washed organic layer was obtained. Next, the water-washed organic layer was filtered to provide filtrate. The resultant filtrate was slowly dropped into methanol (1 L) to provide a precipitate. The precipitate was taken out by filtration. The precipitate thus taken out was dried in a vacuum at a temperature of 70° C. for 12 hours. As a result, a resin (PAR-1) having a viscosity-average molecular weight of 55,000 was obtained.

### Synthesis of Resins (PAR-2) to (PAR-16) and (PAR-R1) to (PAR-R4)

Resins (PAR-2) to (PAR-16) and (PAR-R1) to (PAR-R4) were each synthesized by the same method as that in the synthesis of the resin (PAR-1) except that the molar ratios of the bisphenol and the dicarboxylic acid, and the kind of the end terminator were changed as shown in Table 2. As a result, the resins (PAR-2) to (PAR-16) and (PAR-R1) to (PAR-R4) having viscosity-average molecular weights shown in Table 2 were obtained. The viscosity-average molecular weight of each of the resins (PARs) was adjusted by changing the usage amount of the end terminator, and the usage amount of the end terminator was reduced when the viscosity-average molecular weight of each of the resins (PARs) was increased.

TABLE 2

| | | | Dicarboxylic | | | |
|---|---|---|---|---|---|---|
| | Bisphenol | | acid | | End | Molecular |
| Resin | BP-1 | BP-2 | DC-1 | DC-2 | terminator | weight |
| PAR-1 | 70 | 30 | 65 | 35 | DMP | 55,000 |
| PAR-2 | 70 | 30 | 50 | 50 | DMP | 53,000 |
| PAR-3 | 70 | 30 | 55 | 45 | DMP | 66,000 |
| PAR-4 | 50 | 50 | 80 | 20 | DMP | 79,000 |
| PAR-5 | 50 | 50 | 40 | 60 | DMP | 61,000 |
| PAR-6 | 50 | 50 | 20 | 80 | DMP | 44,000 |
| PAR-7 | 20 | 80 | 65 | 35 | DMP | 56,000 |
| PAR-8 | 20 | 80 | 55 | 45 | DMP | 51,000 |
| PAR-9 | 20 | 80 | 50 | 50 | DMP | 67,000 |
| PAR-10 | 20 | 80 | 65 | 35 | PFH | 53,000 |
| PAR-11 | 10 | 90 | 65 | 35 | DMP | 60,000 |
| PAR-12 | 10 | 90 | 50 | 50 | DMP | 62,000 |
| PAR-13 | 35 | 65 | 30 | 70 | DMP | 61,000 |
| PAR-14 | 50 | 50 | 70 | 30 | DMP | 64,000 |
| PAR-15 | 35 | 65 | 90 | 10 | DMP | 76,000 |
| PAR-16 | 35 | 65 | 75 | 25 | DMP | 72,000 |
| PAR-R1 | 100 | — | 100 | — | DMP | 35,000 |
| PAR-R2 | — | 100 | — | 100 | DMP | 68,000 |
| PAR-R3 | 50 | 50 | — | 100 | DMP | 60,000 |
| PAR-R4 | — | 100 | 50 | 50 | DMP | 55,000 |

The numerical values regarding the bisphenols shown in Table 2 each indicate a molar ratio (percentage) with respect to the total substance amount in terms of mole of the compound (BP-1) and the compound (BP-2). In addition, the numerical values regarding the dicarboxylic acids shown in Table 2 each indicate a molar ratio (percentage) with respect to the total substance amount in terms of mole of the compound (DC-1) and the compound (DC-2). In addition, “PFH” represents 1H,1H-perfluoro-1-heptanol. In addition, the molecular weight indicates a viscosity-average molecular weight.

### Example 1

### <Production of Electrophotographic Photosensitive Member>

### [Production of Photosensitive Member 1]

An aluminum cylinder (JIS-A3003, aluminum alloy) having a length of 260.5 mm and a diameter of 30 mm was subjected to cutting processing (JIS B0601:2014, ten-point average roughness Rzjis: 0.8 μm), and the resultant was used as an electroconductive support.

The following materials were prepared.

| | |
|---|---|
| Y-form titanyl phthalocyanine (CGM-1) servig | 2.0 parts by mass |
| as a charge generating material | |
| Hole transporting material (H-1) | 70.0 parts by mass |
| Electron transporting material (E-4) | 35.0 parts by mass |
| Polyarylate resin (PAR-1) serving as a binder resin | 100.0 parts by mass |
| Additive (T-1) | 1.6 parts by mass |
| Tetrahydrofuran serving as a solvent | 500.0 parts by mass |

The above-mentioned materials were mixed with a rod-shaped sonic oscillator for 20 minutes to provide a dispersion liquid. The dispersion liquid was filtered through a filter having an opening of 5 μm to provide a coating liquid for a photosensitive layer. The coating liquid for a photosensitive layer was applied onto the electroconductive support by a dip coating method, and was dried with hot air at 120° C. for 50 minutes. Thus, a photosensitive layer (thickness: 30 m) was formed on the electroconductive support to provide a photosensitive member 1.

### (Resin Component Analysis of Photosensitive Member 1)

A ¹H-NMR spectrum was obtained by ¹H-nuclear magnetic resonance spectrometry of polymer components recovered from the resultant photosensitive member in deuterated chloroform. The resultant ¹H-NMR spectrum had peaks at 8.22±0.02 ppm, 7.18±0.02 ppm, 7.16±0.02 ppm, 7.10±0.02 ppm, 7.06±0.02 ppm, 7.04±0.02 ppm, 2.28±0.02 ppm, 2.20±0.02 ppm, 1.59±0.02 ppm, and 1.54±0.02 ppm. As a result, it was identified that the photosensitive member had the structural units represented by the formulae (1), (2), (3), and (4). In addition, it was able to be recognized that the ratios of the substance amounts in terms of mole of the structural units represented by the formula (1), the formula (2), the formula (3), and the formula (4) were as shown in Table 2 based on the integration ratios of the above-mentioned peaks.

### <Evaluation>

The photosensitive member (monolayer type photosensitive member) was evaluated for image smearing by the following method.

A modified apparatus obtained by modifying a monochrome laser printer (product name: HL-5200, manufactured by Brother Industries, Ltd.) was used as an electrophotographic apparatus. A high-voltage power supply control system (product name: Model 615-3, manufactured by Trek Japan) was used as a power supply for supplying electric power for a corona charger from the outside of the printer. An electrophotographic photosensitive member of a drum unit in a cartridge for this printer was removed, and the photosensitive member 1 was set instead.

### [Evaluation of Image Smearing]

First, the electrophotographic apparatus and the photosensitive member were left under an environment at a temperature of 30° C. and a humidity of 80% RH for 24 hours or more, and then the photosensitive member was mounted on the electrophotographic apparatus.

The amount of current flowing through a corona wire of the corona charger was adjusted to 500 μA, and an image having an image print ratio of 3% was output onto 30,000 sheets of A4 portrait size paper. After that, the power supply to the electrophotographic apparatus was stopped and deactivated for 3 days. After the deactivation for 3 days, the power supply to the electrophotographic apparatus was started again, and a character image (E character image) in which a lattice image and the alphabetical character E (font type: Times, font size: 6 points) were repeated was output onto A4 portrait size paper. The resultant images were each evaluated for an image defect suppressing effect in accordance with the following evaluation ranks. The higher rank number indicates a more satisfactory effect, and the ranks 5, 4, and 3 were determined to be the levels at which the image defect suppressing effect was obtained. Meanwhile, the ranks 1 and 2 were determined to be the levels at which the image defect suppressing effect was not obtained. The evaluation results are shown in Tables 3-1, 3-2, 3-3 and 3-4.

Rank 5: No image defect is observed in both the lattice image and the E character image.

Rank 4: The lattice image is partially blurred, but no image defect is observed in the E character image.

Rank 3: The lattice image is partially blurred, and the E character image is partially faded.

Rank 2: The lattice image is partially lost, and the E character image is entirely faded.

Rank 1: The lattice image is entirely lost, and the E character image is entirely faded.

### [Evaluation of Wear Resistance]

The thickness on the surface of a center portion of the photosensitive member 1 used in the evaluation of image smearing was measured for a decrease amount (abrasion amount) from an initial stage. In this case, the thickness was measured with a thickness measurement device FISCHER MMS eddy current probe EAW3.3 manufactured by FISCHER INSTRUMENTS K.K. The decrease amount of the thickness of the photosensitive layer was evaluated based on a value obtained by converting the decrease amount after the output of an image onto 30,000 sheets into the decrease amount per 1,000 sheets. The evaluation results are shown in Tables 3-1, 3-2, 3-3 and 3-4.

### Examples 2 to 32 and Comparative Examples 1 to 7

Electrophotographic photosensitive members were each produced and evaluated in the same manner as in Example 1 except that the kinds and amounts of the binder resin, the hole transporting material, the electron transporting material, the charge generating material, and the additive to be mixed in the coating liquid for a photosensitive layer were changed as shown in Tables 3-1, 3-2, 3-3 and 3-4. The results are shown in Tables 3-1, 3-2, 3-3 and 3-4.

The hole transporting material HR-1 shown in Tables 3-1, 3-2, 3-3 and 3-4 is a compound represented by the following formula (HR-1).

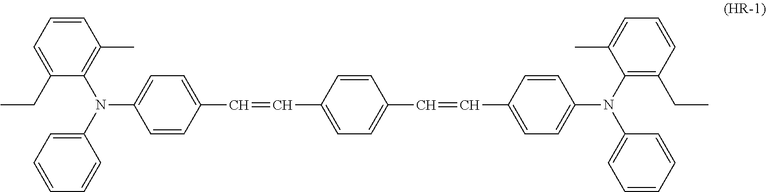 [SMILES: CCc1cccc(C)c1N(c1ccccc1)c1ccc(C=Cc2ccc(C=Cc3ccc(N(c4ccccc4)c4c(C)cccc4CC)cc3)cc2)cc1]

In addition, the hole transporting material HR-2 shown in Tables 3-1, 3-2, 3-3 and 3-4 is a compound represented by the following formula (HR-2).

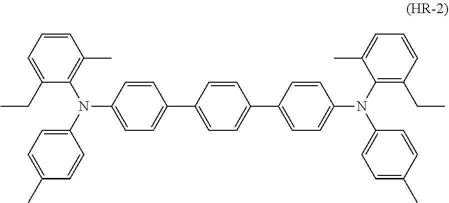 [SMILES: CCc1cccc(C)c1N(c1ccc(C)cc1)c1ccc(-c2ccc(-c3ccc(N(c4ccc(C)cc4)c4c(C)cccc4CC)cc3)cc2)cc1]

In addition, the hole transporting material HR-3 shown in Tables 3-1, 3-2, 3-3 and 3-4 is a compound represented by the following formula (HR-3).

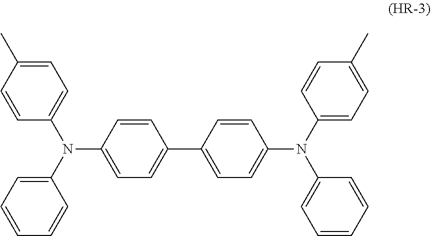 [SMILES: Cc1ccc(N(c2ccccc2)c2ccc(-c3ccc(N(c4ccccc4)c4ccc(C)cc4)cc3)cc2)cc1]

TABLE 3-1

| | | | | Hole transporting | | Electron transporting | |
|---|---|---|---|---|---|---|---|
| | | Polyarylate resin | | material | | material | |
| | Photosensitive | | Parts by | | Parts by | | Parts by |
| | member | Kind | mass | Kind | mass | Kind | mass |
| Example 1 | Photosensitive | PAR-1 | 100 | H-1 | 70 | E-4 | 35 |
| | member 1 | | | | | | |
| Example 2 | Photosensitive | PAR-2 | 100 | H-1 | 70 | E-4 | 35 |
| | member 2 | | | | | | |
| Example 3 | Photosensitive | PAR-3 | 100 | H-1 | 70 | E-4 | 35 |
| | member 3 | | | | | | |
| Example 4 | Photosensitive | PAR-4 | 100 | H-1 | 70 | E-4 | 35 |
| | member 4 | | | | | | |
| Example 5 | Photosensitive | PAR-5 | 100 | H-1 | 70 | E-4 | 35 |
| | member 5 | | | | | | |
| Example 6 | Photosensitive | PAR-6 | 100 | H-1 | 70 | E-4 | 35 |
| | member 6 | | | | | | |
| Example 7 | Photosensitive | PAR-7 | 100 | H-1 | 70 | E-4 | 35 |
| | member 7 | | | | | | |
| Example 8 | Photosensitive | PAR-8 | 100 | H-1 | 70 | E-4 | 35 |
| | member 8 | | | | | | |
| Example 9 | Photosensitive | PAR-9 | 100 | H-1 | 70 | E-4 | 35 |
| | member 9 | | | | | | |
| Example 10 | Photosensitive | PAR-10 | 100 | H-1 | 70 | E-4 | 35 |
| | member 10 | | | | | | |
| Example 11 | Photosensitive | PAR-11 | 100 | H-1 | 70 | E-4 | 35 |
| | member 11 | | | | | | |
| Example 12 | Photosensitive | PAR-12 | 100 | H-1 | 70 | E-4 | 35 |
| | member 12 | | | | | | |
| Example 13 | Photosensitive | PAR-7 | 70 | H-1 | 70 | E-4 | 35 |
| | member 13 | | | | | | |
| Example 14 | Photosensitive | PAR-7 | 120 | H-1 | 70 | E-4 | 35 |
| | member 14 | | | | | | |
| Example 15 | Photosensitive | PAR-7 | 100 | H-1 | 55 | E-4 | 50 |
| | member 15 | | | | | | |
| Example 16 | Photosensitive | PAR-7 | 100 | H-1 | 80 | E-4 | 25 |
| | member 16 | | | | | | |
| Example 17 | Photosensitive | PAR-7 | 100 | H-2 | 70 | E-4 | 35 |
| | member 17 | | | | | | |
| Example 18 | Photosensitive | PAR-7 | 100 | H-3 | 70 | E-4 | 35 |
| | member 18 | | | | | | |
| Example 19 | Photosensitive | PAR-7 | 100 | H-5 | 70 | E-4 | 35 |
| | member 19 | | | | | | |
| Example 20 | Photosensitive | PAR-7 | 100 | H-6 | 70 | E-4 | 35 |
| | member 20 | | | | | | |
| Example 21 | Photosensitive | PAR-7 | 100 | H-7 | 70 | E-4 | 35 |
| | member 21 | | | | | | |
| Example 22 | Photosensitive | PAR-7 | 100 | H-9 | 70 | E-4 | 35 |
| | member 22 | | | | | | |
| Example 23 | Photosensitive | PAR-7 | 100 | H-1 | 70 | E-1 | 35 |
| | member 23 | | | | | | |
| Example 24 | Photosensitive | PAR-7 | 100 | H-1 | 70 | E-2 | 35 |
| | member 24 | | | | | | |
| Example 25 | Photosensitive | PAR-7 | 100 | H-1 | 70 | E-3 | 35 |
| | member 25 | | | | | | |

TABLE 3-2

| | | | | Hole transporting | | Electron transporting | |
|---|---|---|---|---|---|---|---|
| | | Polyarylate resin | | material | | material | |
| | Photosensitive | | Parts by | | Parts by | | Parts by |
| | member | Kind | mass | Kind | mass | Kind | mass |
| Example 26 | Photosensitive | PAR-7 | 100 | H-1 | 70 | E-5 | 35 |
| | member 26 | | | | | | |
| Example 27 | Photosensitive | PAR-7 | 100 | H-1 | 70 | E-6 | 35 |
| | member 27 | | | | | | |
| Example 28 | Photosensitive | PAR-7 | 100 | H-1 | 70 | E-7 | 35 |
| | member 28 | | | | | | |
| Example 29 | Photosensitive | PAR-7 | 100 | H-1 | 70 | E-8 | 35 |
| | member 29 | | | | | | |
| Example 30 | Photosensitive | PAR-7 | 100 | H-1 | 70 | E-4 | 35 |
| | member 30 | | | | | | |
| Example 31 | Photosensitive | PAR-7 | 100 | H-1 | 70 | E-4 | 35 |
| | member 31 | | | | | | |
| Example 32 | Photosensitive | PAR-7 | 100 | H-1 | 70 | E-4 | 35 |
| | member 32 | | | | | | |
| Example 33 | Photosensitive | PAR-7 | 100 | H-1 | 70 | E-4 | 35 |
| | member 33 | | | | | | |
| Example 34 | Photosensitive | PAR-13 | 100 | H-1 | 70 | E-4 | 35 |
| | member 34 | | | | | | |
| Example 35 | Photosensitive | PAR-14 | 100 | H-1 | 70 | E-4 | 35 |
| | member 35 | | | | | | |
| Example 36 | Photosensitive | PAR-15 | 100 | H-1 | 70 | E-4 | 35 |
| | member 36 | | | | | | |
| Example 37 | Photosensitive | PAR-16 | 100 | H-1 | 70 | E-4 | 35 |
| | member 37 | | | | | | |
| Example 38 | Photosensitive | PAR-7/ | 60/40 | H-1 | 70 | E-4 | 35 |
| | member 38 | PAR-R3 | | | | | |
| Example 39 | Photosensitive | PAR-7/ | 50/50 | H-1 | 70 | E-4 | 35 |
| | member 39 | PAR-R3 | | | | | |
| Example 40 | Photosensitive | PAR-7/ | 40/60 | H-1 | 70 | E-4 | 35 |
| | member 40 | PAR-R3 | | | | | |
| Example 41 | Photosensitive | PAR-7 | 100 | H-1/ | 35/35 | E-4 | 35 |
| | member 41 | | | HR-3 | | | |
| Example 42 | Photosensitive | PAR-7 | 100 | H-1/ | 42/28 | E-4 | 35 |
| | member 42 | | | HR-3 | | | |
| Comparative | Photosensitive | PAR-R1 | 100 | H-1 | 70 | E-4 | 35 |
| Example 1 | member R1 | | | | | | |
| Comparative | Photosensitive | PAR-R2 | 100 | H-1 | 70 | E-4 | 35 |
| Example 2 | member R2 | | | | | | |
| Comparative | Photosensitive | PAR-R3 | 100 | H-1 | 70 | E-4 | 35 |
| Example 3 | member R3 | | | | | | |
| Comparative | Photosensitive | PAR-R4 | 100 | H-1 | 70 | E-4 | 35 |
| Example 4 | member R4 | | | | | | |
| Comparative | Photosensitive | PAR-1 | 100 | HR-1 | 70 | E-4 | 35 |
| Example 5 | member R5 | | | | | | |
| Comparative | Photosensitive | PAR-1 | 100 | HR-2 | 70 | E-4 | 35 |
| Example 6 | member R6 | | | | | | |
| Comparative | Photosensitive | PAR-1 | 100 | HR-3 | 70 | E-4 | 35 |
| Example 7 | member R7 | | | | | | |

TABLE 3-3

| | Charge generating material | | Additive | | Evaluation of | Abrasion |
|---|---|---|---|---|---|---|
| | | Parts by | | Parts by | image | amount |
| | Kind | mass | Kind | mass | smearing | (μm) |
| Example 1 | CGM-1 | 2 | T-1 | 1.6 | 5 | 1.2 |
| Example 2 | CGM-1 | 2 | T-1 | 1.6 | 5 | 1.5 |
| Example 3 | CGM-1 | 2 | T-1 | 1.6 | 5 | 1.3 |
| Example 4 | CGM-1 | 2 | T-1 | 1.6 | 5 | 1.3 |
| Example 5 | CGM-1 | 2 | T-1 | 1.6 | 5 | 1.4 |
| Example 6 | CGM-1 | 2 | T-1 | 1.6 | 5 | 1.6 |
| Example 7 | CGM-1 | 2 | T-1 | 1.6 | 5 | 0.9 |
| Example 8 | CGM-1 | 2 | T-1 | 1.6 | 5 | 0.9 |
| Example 9 | CGM-1 | 2 | T-1 | 1.6 | 5 | 1.1 |
| Example 10 | CGM-1 | 2 | T-1 | 1.6 | 5 | 1.0 |
| Example 11 | CGM-1 | 2 | T-1 | 1.6 | 5 | 1.4 |
| Example 12 | CGM-1 | 2 | T-1 | 1.6 | 5 | 1.5 |
| Example 13 | CGM-1 | 2 | T-1 | 1.6 | 5 | 1.1 |
| Example 14 | CGM-1 | 2 | T-1 | 1.6 | 5 | 0.8 |
| Example 15 | CGM-1 | 2 | T-1 | 1.6 | 5 | 1.1 |
| Example 16 | CGM-1 | 2 | T-1 | 1.6 | 5 | 1.0 |
| Example 17 | CGM-1 | 2 | T-1 | 1.6 | 5 | 1.1 |
| Example 18 | CGM-1 | 2 | T-1 | 1.6 | 5 | 1.2 |
| Example 19 | CGM-1 | 2 | T-1 | 1.6 | 5 | 1.5 |
| Example 20 | CGM-1 | 2 | T-1 | 1.6 | 5 | 1.5 |
| Example 21 | CGM-1 | 2 | T-1 | 1.6 | 5 | 1.3 |
| Example 22 | CGM-1 | 2 | T-1 | 1.6 | 5 | 1.4 |
| Example 23 | CGM-1 | 2 | T-1 | 1.6 | 5 | 1.3 |
| Example 24 | CGM-1 | 2 | T-1 | 1.6 | 5 | 1.4 |
| Example 25 | CGM-1 | 2 | T-1 | 1.6 | 5 | 1.2 |

TABLE 3-4

| | Charge generating material | | Additive | | Evaluation of | Amount of |
|---|---|---|---|---|---|---|
| | | Parts by | | Parts by | image | abrasion |
| | Kind | mass | Kind | mass | smearing | (μm) |
| Example 26 | CGM-1 | 2 | T-1 | 1.6 | 5 | 1.6 |
| Example 27 | CGM-1 | 2 | T-1 | 1.6 | 5 | 1.5 |
| Example 28 | CGM-1 | 2 | T-1 | 1.6 | 5 | 1.3 |
| Example 29 | CGM-1 | 2 | T-1 | 1.6 | 5 | 1.6 |
| Example 30 | CGM-1 | 2 | T-2 | 1.6 | 5 | 1.0 |
| Example 31 | CGM-1 | 2 | T-3 | 1.6 | 5 | 1.1 |
| Example 32 | CGM-1 | 2 | T-4 | 1.6 | 5 | 0.9 |
| Example 33 | CGM-1 | 2 | None | 0 | 5 | 1.2 |
| Example 34 | CGM-1 | 2 | T-1 | 1.6 | 5 | 1.4 |
| Example 35 | CGM-1 | 2 | T-1 | 1.6 | 5 | 0.8 |
| Example 36 | CGM-1 | 2 | T-1 | 1.6 | 5 | 1.3 |
| Example 37 | CGM-1 | 2 | T-1 | 1.6 | 5 | 1.6 |
| Example 38 | CGM-1 | 2 | T-1 | 1.6 | 5 | 1.2 |
| Example 39 | CGM-1 | 2 | T-1 | 1.6 | 5 | 1.5 |
| Example 40 | CGM-1 | 2 | T-1 | 1.6 | 5 | 1.8 |
| Example 41 | CGM-1 | 2 | T-1 | 1.6 | 5 | 1.9 |
| Example 42 | CGM-1 | 2 | T-1 | 1.6 | 5 | 1.5 |
| Comparative | CGM-1 | 2 | T-1 | 1.6 | 1 | 0.8 |
| Example 1 | | | | | | |
| Comparative | CGM-1 | 2 | T-1 | 1.6 | 5 | 2.5 |
| Example 2 | | | | | | |
| Comparative | CGM-1 | 2 | T-1 | 1.6 | 3 | 2.1 |
| Example 3 | | | | | | |
| Comparative | CGM-1 | 2 | T-1 | 1.6 | 1 | 1.8 |
| Example 4 | | | | | | |
| Comparative | CGM-1 | 2 | T-1 | 1.6 | 2 | 1.7 |
| Example 5 | | | | | | |
| Comparative | CGM-1 | 2 | T-1 | 1.6 | 2 | 2.0 |
| Example 6 | | | | | | |
| Comparative | CGM-1 | 2 | T-1 | 1.6 | 2 | 1.9 |
| Example 7 | | | | | | |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-195280, filed Nov. 16, 2023, and Japanese Patent Application No. 2024-173827, filed Oct. 2, 2024, which are hereby incorporated by reference herein in their entirety.

What is claimed is:

1. An electrophotographic photosensitive member comprising:
a support; and
a photosensitive layer,
wherein the photosensitive layer contains a binder resin, a charge generating material, an electron transporting material, and a hole transporting material,
wherein the binder resin contains a polyarylate resin having a structural unit represented by the following formula (1), a structural unit represented by the following formula (2), a structural unit represented by the following formula (3), and a structural unit represented by the following formula (4): 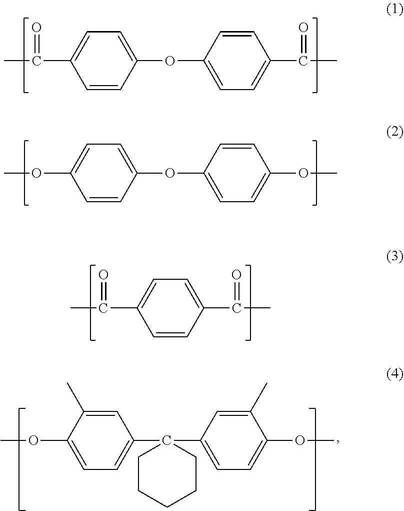
and
wherein the hole transporting material contains a compound represented by the following formula (5): 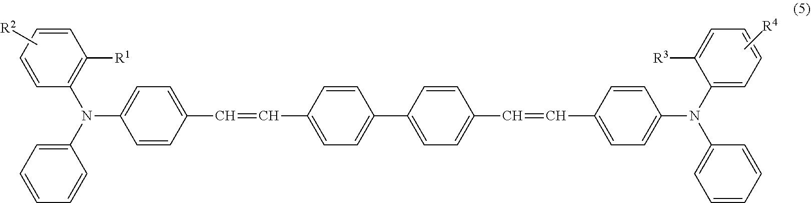
in the formula (5), R¹, R², R³ and R⁴ each independently represent an alkyl group having 1 or more and 4 or less carbon atoms, or an alkoxy group having 1 or more and 4 or less carbon atoms.

2. The electrophotographic photosensitive member according to claim 1, wherein a ratio of a substance amount in terms of mole of the structural unit represented by the formula (1) is 0.30 or more with respect to a total substance amount in terms of mole of structural units derived from dicarboxylic acids for forming the polyarylate resin.

3. The electrophotographic photosensitive member according to claim 1, wherein, in the polyarylate resin, when a ratio of a substance amount in terms of mole of the structural unit represented by the formula (1) to a total substance amount in terms of mole of the structural units for forming the polyarylate resin is represented by M1, a ratio of a substance amount in terms of mole of the structural unit represented by the formula (2) to the total substance amount in terms of mole of the structural units for forming the polyarylate resin is represented by M2, a ratio of a substance amount in terms of mole of the structural unit represented by the formula (3) to the total substance amount in terms of mole of the structural units for forming the polyarylate resin is represented by M3, and a ratio of a substance amount in terms of mole of the structural unit represented by the formula (4) to the total substance amount in terms of mole of the structural units for forming the polyarylate resin is represented by M4, M3/(M1+M3) is 0.30 or more and less than 0.70, and M2/(M2+M4) is 0.10 or more and less than 0.50.

4. The electrophotographic photosensitive member according to claim 1, wherein a ratio of the polyarylate resin is 50 mass % or more with respect to a total mass of the binder resin.

5. The electrophotographic photosensitive member according to claim 1, wherein a content of the hole transporting material is 50 parts by mass or more and 90 parts by mass or less with respect to 100 parts by mass of the binder resin.

6. The electrophotographic photosensitive member according to claim 1, wherein a ratio of a substance amount in terms of mole of the compound represented by the formula (5) is 0.6 or more with respect to a total substance amount in terms of mole of the hole transporting material.

7. The electrophotographic photosensitive member according to claim 1, wherein, in the formula (5), R¹ and R³ represent groups identical to each other, and R² and R⁴ represent groups identical to each other.

8. The electrophotographic photosensitive member according to claim 1, wherein, in the formula (5), substitution positions of R² and R⁴ are each a 6-position.

9. The electrophotographic photosensitive member according to claim 1, wherein the electron transporting material contains at least one compound selected from the group consisting of: a compound represented by the following formula (6); a compound represented by the following formula (7); a compound represented by the following formula (8); a compound represented by the following formula (9); a compound represented by the following formula (10); a compound represented by the following formula (11); and a compound represented by the following formula (12): 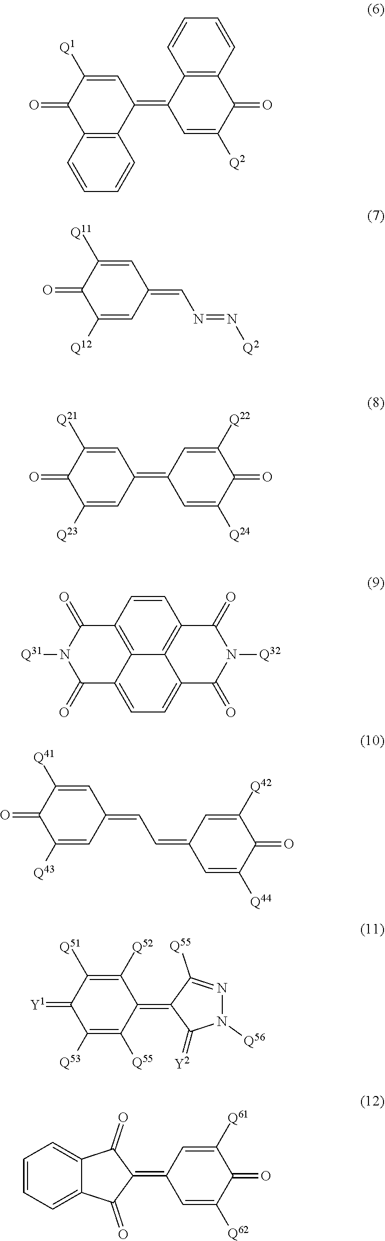
where Q¹ and Q² in the formula (6), Q¹¹, Q¹², and Q¹³ in the formula (7), Q²¹, Q²², Q²³, and Q²⁴ in the formula (8), Q³¹ and Q³² in the formula (9), Q⁴¹, Q⁴², Q⁴³, and Q⁴⁴ in the formula (10), Q⁵¹, Q⁵², Q⁵³, Q⁵⁴, Q⁵⁵, and Q⁵⁶ in the formula (11), and Q⁶¹ and Q⁶² in the formula (12) each independently represent a hydrogen atom, a halogen atom, a cyano group, an alkyl group having 1 or more and 6 or less carbon atoms, an alkenyl group having 2 or more and 6 or less carbon atoms, an alkoxy group having 1 or more and 6 or less carbon atoms, or an aryl group having 6 or more and 14 or less carbon atoms that may be substituted with at least one substituent selected from the group consisting of: an alkyl group having 1 or more and 6 or less carbon atoms; and a halogen atom, and Y¹ and Y² in the formula (11) each independently represent an oxygen atom or a sulfur atom.

10. The electrophotographic photosensitive member according to claim 9, wherein the electron transporting material contains at least one compound selected from the group consisting of: a compound represented by the following formula (E-1); a compound represented by the following formula (E-2); a compound represented by the following formula (E-3); a compound represented by the following formula (E-4); a compound represented by the following formula (E-5); a compound represented by the following formula (E-6); a compound represented by the following formula (E-7); and a compound represented by the following formula (E-8).

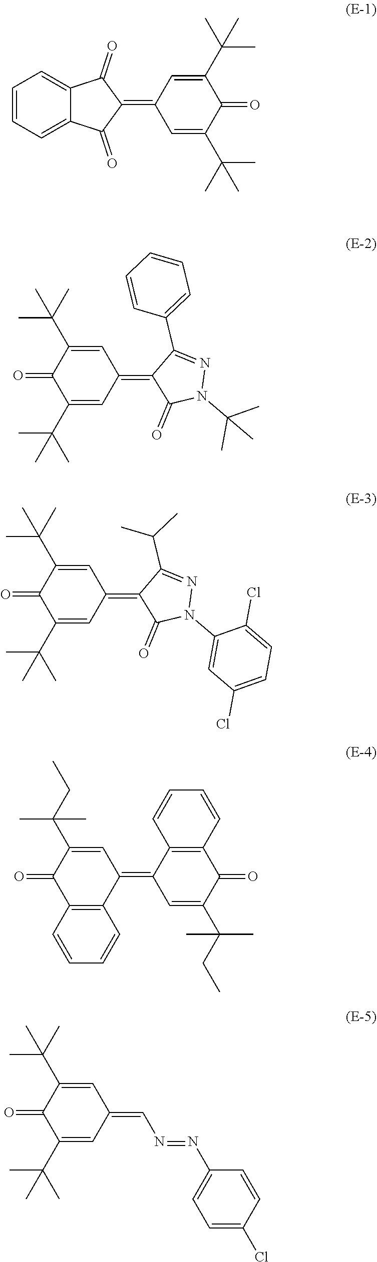

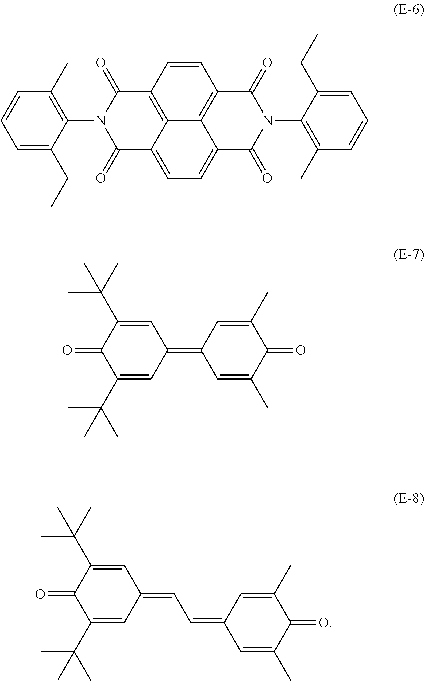 [SMILES: CCc1cccc(C)c1N1C(=O)c2ccc3c4c(ccc(c24)C1=O)C(=O)N(c1c(C)cccc1CC)C3=O ; CC1=CC(=C2C=C(C(C)(C)C)C(=O)C(C(C)(C)C)=C2)C=C(C)C1=O ; CC1=CC(=CC=C2C=C(C(C)(C)C)C(=O)C(C(C)(C)C)=C2)C=C(C)C1=O]

11. The electrophotographic photosensitive member according to claim 1, wherein the photosensitive layer contains, as an additive, at least one compound selected from the group consisting of: a compound represented by the following formula (T-1); a compound represented by the following formula (T-2); a compound represented by the following formula (T-3); and a compound represented by the following formula (T-4).

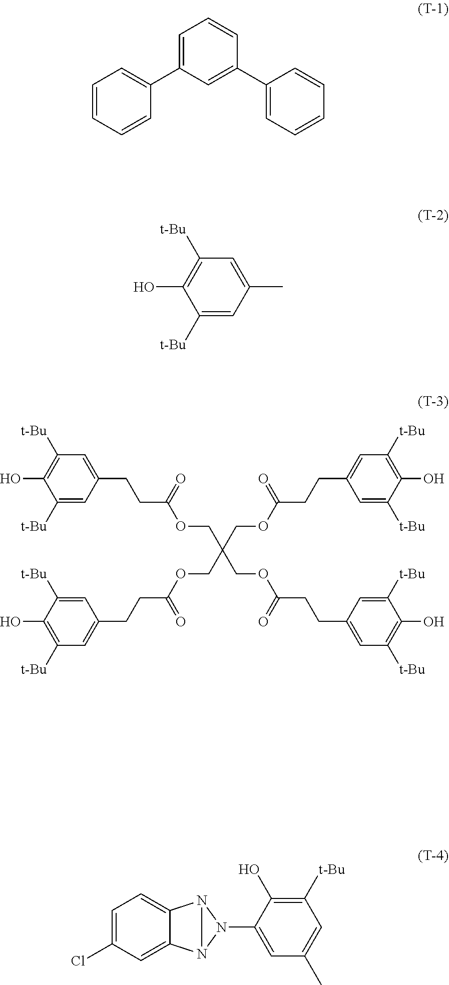

12. A process cartridge comprising:
an electrophotographic photosensitive member; and at least one unit selected from the group consisting of: a charging unit; a developing unit; and a cleaning unit,
the process cartridge integrally supporting the electrophotographic photosensitive member and the at least one unit, and being detachably attachable onto a main body of an electrophotographic apparatus,
the electrophotographic photosensitive member comprising:
a support; and
a photosensitive layer,
wherein the photosensitive layer contains a binder resin, a charge generating material, an electron transporting material, and a hole transporting material,
wherein the binder resin contains a polyarylate resin having a structural unit represented by the following formula (1), a structural unit represented by the following formula (2), a structural unit represented by the following formula (3), and a structural unit represented by the following formula (4): 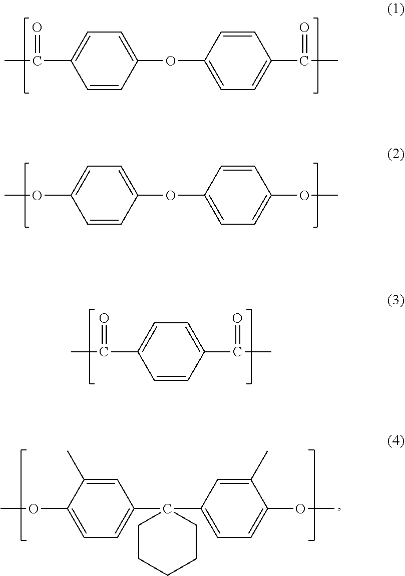
and
wherein the hole transporting material contains a compound represented by the following formula (5): 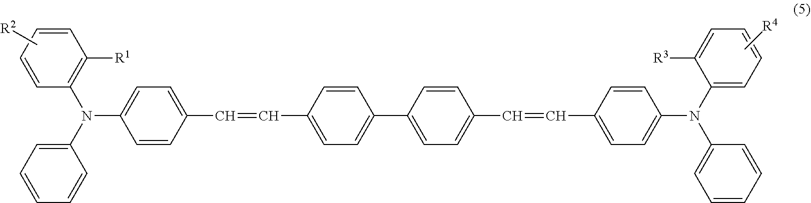
in the formula (5), R¹, R², R³, and R⁴ each independently represent an alkyl group having 1 or more and 4 or less carbon atoms, or an alkoxy group having 1 or more and 4 or less carbon atoms.

13. The process cartridge according to claim 12,
wherein the process cartridge comprises the charging unit, and
wherein the charging unit is a charging unit configured to positively charge the electrophotographic photosensitive member.

14. An electrophotographic apparatus comprising:
an electrophotographic photosensitive member;
a charging unit;
an exposing unit;
a developing unit; and
a transfer unit,
the electrophotographic photosensitive member comprising:
a support; and
a photosensitive layer,
wherein the photosensitive layer contains a binder resin, a charge generating material, an electron transporting material, and a hole transporting material,
wherein the binder resin contains a polyarylate resin having a structural unit represented by the following formula (1), a structural unit represented by the following formula (2), a structural unit represented by the following formula (3), and a structural unit represented by the following formula (4): 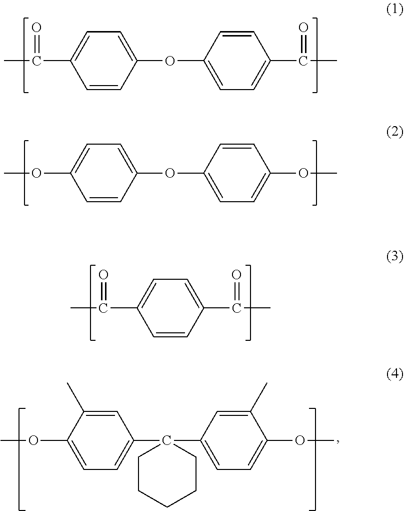
and
wherein the hole transporting material contains a compound represented by the following formula (5): 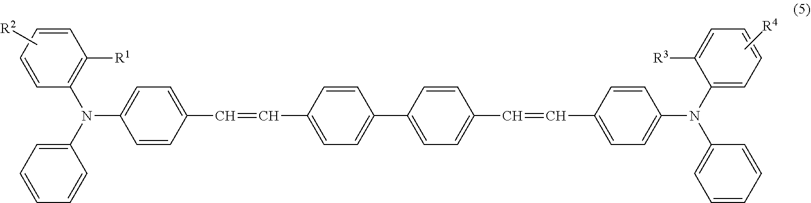
in the formula (5), R¹, R², R³, and R⁴ each independently represent an alkyl group having 1 or more and 4 or less carbon atoms, or an alkoxy group having 1 or more and 4 or less carbon atoms.

15. The electrophotographic apparatus according to claim 14, wherein the charging unit is a charging unit configured to positively charge the electrophotographic photosensitive member.
